(19) United States
(12) Patent Application Publication
(10) Pub. No.: US 2008/0167552 A1
(43) Pub. Date: Jul. 10, 2008

(54) SYSTEM AND METHOD OF GENERATING AN IMAGE OF A CONTRAST AGENT INJECTED INTO AN IMAGED SUBJECT

(75) Inventors: Xavier Bouchevreau, Issy-Les-Moulineaux (FR); Serge L.W. Muller, Guyancourt (FR); Razvan G. Iordache, Paris (FR); Fanny Patoureaux, Saint-Cyr L'Ecole (FR)

Correspondence Address: PETER VOGEL;GE HEALTHCARE, 3000 N. GRANDVIEW BLVD., SN-477, WAUKESHA, WI, 53188, US

(73) Assignee: GENERAL ELECTRIC COMPANY, Schenectady, NY (US)

(21) Appl. No.: 11/619,804

(22) Filed: Jan. 4, 2007

(51) Int. Cl. A61B 5/00

(52) U.S. Cl. 600/431; 382/130

(57) ABSTRACT

An imaging system operable to generate an output image of a contrast agent injected in an imaged subject. The system includes a energy source, a detector, and a display. The detector generates a plurality of radiological images of the imaged subject. The system also includes a computer having a memory in communication with a processor. The memory includes programmable instructions, including acquiring an image of the contrast agent in the imaged subject with a spectra of energy from the energy source; detecting grayscale values of pixel data of the contrast agent in the image; calculating a predicted thickness of the contrast agent relative to the plurality of grayscale values of pixel data of the contrast agent detected in the image; and generating an output image comprising an illustration of the predicted thickness of the contrast agent for illustration on the display.

### BACKGROUND OF THE INVENTION

The subject matter described herein generally relates to medical imaging, and more particularly to a system and method generating an image of a contrast agent injected in an imaged subject, such as employed in mammography to analyze breast tissue.

Dual energy acquisition is a certain known method used to perform diagnostic mammography. This certain method of dual energy acquisition includes injecting the breast tissue of an imaged subject with a contrast agent (e.g. iodine), and acquiring a pair of images with differing spectras or ranges of energy (e.g., spectras of X rays). Referring to FIG. 1, one way of choosing the different spectra or range of energy is to select spectra from each side of a K-edge 20 of the injected contrast agent. The K-edge 20 is generally an increase in an attenuation coefficient of photons occurring at a photon energy just above a binding energy of an electron at the K-shell of atoms interacting with the photons. Contrast agents such as iodine or barium have K-shell binding energies (about 33.2 keV and 37.4 keV, respectively) with enhanced absorption of X-ray radiation.

In accordance with this certain known method of dual energy acquisition, a first image, called the low-energy image, is acquired with an energy range lower than the K-edge 20 of iodine (about 33.2 keV) (See FIG. 1). With the first image, the differential attenuation of radiation produced by the injected contrast agent in the breast will be relatively low and will generally demonstrate a high contrast between adipose and glandular type breast tissues. The second image, called the high-energy image, is acquired with an energy range or spectra higher than relative to the K-edge 20 of the contrast agent. Accordingly, in comparison to the first image, the second image includes a higher differential attenuation of radiation produced by the injected contrast agent n the breast tissue and such that the second image generally demonstrates a higher contrast between the contrast agent and the breast tissues. However, it is known that with the image data acquired using dual energy acquisition, the contrast agent may not be clearly visible even when using a well-suited spectrum of energy to acquire the image of the injected contrast agent.

The certain known method of dual energy image acquisition includes a known logarithmic subtraction technique, in the form S=log(xₕ)−Rlog(xₗ) where (S) is the subtracted image and (Xₕ) and (xₗ) are the grayscale values of pixel data in the high-energy image and in the low-energy image, respectively. If the spectra of energy used acquire the images is mono-energetic, adjusting the parameter (R) to a well-suited value can suppress or subtract undesired image data associated with the breast tissue, leaving the image data of the contrast agent. However, it is known that this certain known method of logarithmic subtraction is not suitable when the spectra of energy used acquire the images is not mono-energetic.

Thus, there is need for a system and method of dual energy image reconstruction with enhanced visualization of the injected contrast agent that addresses the drawbacks described above. For example, there is a need for a system to reduce structure noise in the generated reconstructed images where the spectra are not mono-energetic or where the breast tissue composition is not uniform because of the spatial repartition of the glandular and fat tissues in the breast tissue.

### BRIEF DESCRIPTION OF THE INVENTION

The above-mentioned drawbacks are addressed by the embodiments of the subject matter described herein.

In accordance with one embodiment, an imaging system operable to generate an output image of a contrast agent injected into an imaged subject is provided. The system includes an energy source in communication with a detector, the detector operable to generate a plurality of radiological images of the imaged subject injected with the contrast agent. The system also includes a computer in communication with a display and to receive the acquired plurality of images from the detector. The computer includes a memory in communication with a processor, the memory including a plurality of programmable instructions for execution by the processor. The plurality of programmable instructions include acquiring at least one image of the contrast agent in the imaged subject with a spectra of energy from the energy source; detecting a plurality of grayscale values of pixel data of the contrast agent in the at least one image; calculating a predicted thickness of the contrast agent relative to the plurality of grayscale values of pixel data of the contrast agent detected in the at least one image; and generating an output image comprising an illustration of the predicted thickness of the contrast agent for illustration on the display.

In accordance with another embodiment, a method of generating an output image illustrative of a contrast agent injected into an imaged subject is provided. The method comprising the acts of acquiring at least one radiological image of the imaged subject under a spectra of energy; detecting a plurality of grayscale values of pixel data of the contrast agent in the first and second images; calculating a predicted thickness of the contrast agent relative to the plurality of grayscale values of pixel data of the contrast agent detected in the first and second images; and generating an output image comprising an illustration of the predicted thickness of the contrast agent for illustration on the display.

In accordance with another embodiment, a calibration phantom to be imaged by a radiological imaging system is provided. The phantom includes a main material of at least one thickness; and at least one insert of a contrast agent of a predetermined thickness located in the main material, the contrast agent operable to be detected in a radiological image of the calibration phantom.

Embodiments of varying scope are described herein. In addition to the aspects and advantages described in this summary, further aspects and advantages will become apparent by reference to the drawings and with reference to the detailed description that follows.

### BRIEF DESCRIPTION OF THE DRAWINGS

FIG. 1 illustrates a schematic diagram of a K-edge of a contrast agent known in the art.

FIG. 2 shows a schematic diagram of one embodiment of an imaging system operable to generate an image of a contrast agent injected into an imaged subject.

FIG. 3 illustrates a block diagram of one embodiment of a method of generating an image of an injected contrast agent injected into an imaged subject.

FIG. 4 illustrates a schematic diagram of an embodiment of a technique to thicken a border of region of interest in an output image.

FIG. 5 illustrates a schematic diagram of a cross-sectional view of an embodiment of a calibration phantom with a series of inserts of contrast agent of varying thickness.

FIG. 6 illustrates an example of a table of reference points of grey-scale values of pixel data plotted in logarithmic scale relative to a predicted thickness of a contrast agent.

FIG. 7 illustrates a schematic flow diagram of an embodiment of a method to simulate a predicted thickness of contrast agent.

### DETAILED DESCRIPTION OF THE INVENTION

In the following detailed description, reference is made to the accompanying drawings that form a part hereof, and in which is shown by way of illustration specific embodiments, which may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken in a limiting sense.

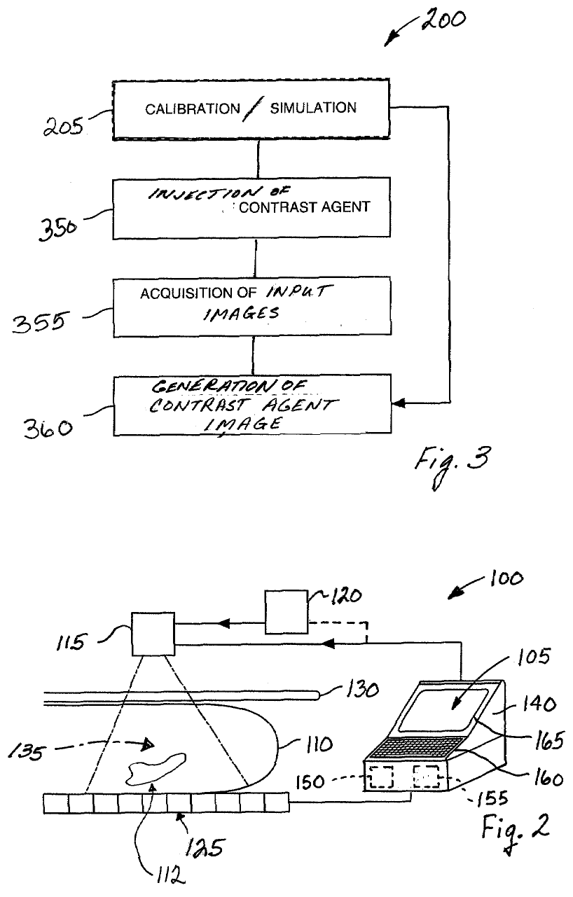

FIG. 2 illustrates one embodiment of a system 100 operable to acquire and generate an output image 105 representative of a contrast agent injected into an imaged subject 110. Assume for sake of example, the imaged subject 110 injected (e.g., via intravenous injection) with a contrast agent 112 (See FIG. 2). Yet, other types of contrast agents 112 can be used.

The system 100 generally includes an energy source 115 (e.g., an X ray source), a controller 120 for controlling an output energy of the source 105, an array of digital detectors 125 for acquiring input images of the imaged subject 110. In the certain example of mammography, the system 100 also includes a plate 130 operable to compress a breast tissue of the imaged subject 110 for enhanced imaging.

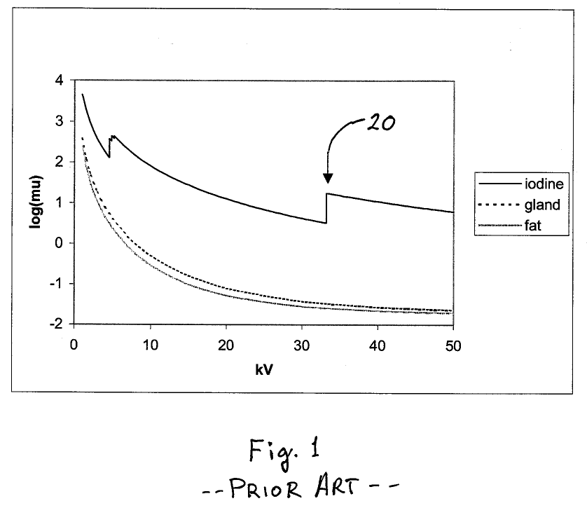

Still referring to FIG. 2, an embodiment of the energy source 115 of the system 100 is configured to acquire at least two input images of a region of interest (ROI) (illustrated by dashed line and reference 135) of the imaged subject using a spectra of different energies, including images acquired with low-energy (i.e., lower than the K-Edge 20 (See FIG. 1) of the contrast agent 112), and with high-energy (i.e., higher than the K-Edge 20 (See FIG. 1) of the contrast agent 112).

The system 100 also includes a computer 140 in communication to receive the acquired images from the array of detectors 125. One embodiment of the computer 140 is also connected in communication with the controller 120 and/or the source 115. The computer 140 is generally configured to process the acquired input images so as to construct or generate an output image 105 with enhanced visualized contrast of the injected contrast agent 112. An embodiment of the computer 140 generally includes a processor 150 operable to execute program instructions stored in a memory 155. The memory 155 can include any type of conventional storage medium (e.g., disk, hard-drive, network database, etc.). The computer also includes an input 160 and an output 165. The input 160 can include a keyboard, a touch-screen, etc. or other known type of input device operable to communicate information to the computer 140. The output 165 can include a monitor, a touch-screen, etc. operable to illustrate the output image 105 generated by the computer 140.

Having described the general construction of the system 100 to generate the output image 105 illustrative of reconstructed thickness of the contrast agent 112 injected in the imaged subject 110, the following is a description of a method 200 (See FIG. 3) of generating the output image 105 of the contrast agent 112 injected into the imaged subject 104. It should be understood that the foregoing sequence of acts comprising the method 200 can vary, that the method 200 may not include each every act in the following description, and the method 200 can include additional acts not disclosed in the following description. One or more of the following acts comprising the method 200 can be represented as computer-readable programmable instructions for storage in the memory 155 and for execution by the processor 150, or stored on a portable computer readable medium such as a floppy disk or CD-ROM for execution by the computer 140.

A technical effect of the system 100 and method 200 described herein generally includes generating the output image 105 of a predicted thickness of the contrast agent 112 injected into a given tissue (e.g., breast) of the ROI 135 of the imaged subject 110 dependent on a detected grayscale value of the pixel data of the acquired input images of the ROI 135 of the imaged subject 110, a thickness of the imaged tissue, a type of imaged tissue (e.g., percentage of glandular to fatty tissue), and the spectras of low-energy and high-energy used to acquire the input images.

Assume, for sake of example, that the contrast agent 112 includes Iodine having a K-edge of 33.2 kV (See FIG. 1). Referring to FIG. 3, act 205 generally includes calibrating or simulating a predicted thickness of the injected contrast agent 112 in the tissue in the ROI 135. The calibration or simulation act 205 allows the computer 140 to calculate the predicted thickness of the contrast agent 112 from input data of detected grayscale levels of pixel data in the acquired input images for illustration in the output display 105 (See FIG. 2).

An embodiment of the calibrating or simulating act 205 includes acquiring input images at low and high-energy of a calibration phantom 230 (See FIG. 4), and generating and storing an algorithm linking, mapping, or correlating predetermined thicknesses of inserts 232 of the contrast agent 112 as a function of the detected grayscale levels of the pixel data acquired in the low and high-energy input images.

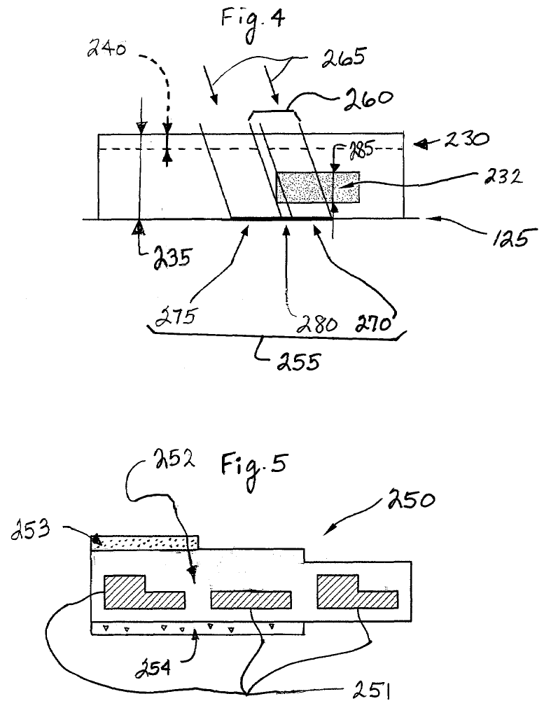

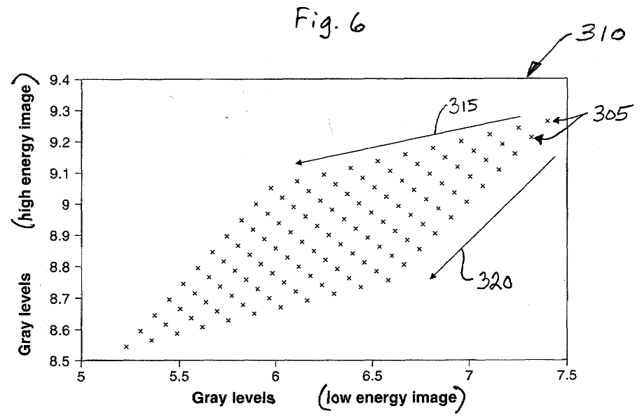

Referring to FIG. 4, an embodiment of the calibration phantom 230 includes predetermined thicknesses of the contrast agent 112 located in predetermined thicknesses of the phantom 230. The embodiment of the calibration phantom 230 is generally comprised of plastic material (e.g., polymethyl methacrylate (PMMA)), with one or more inserts 232 of the contrast agent 112. The calibration phantom 230 can also include one or more predefined layers 240 of material representative of tissue of a specific percentage of glandular tissue 240 on either or both sides of the calibration phantom 230. Adjusting the thickness of the material layer 240 generally acts as an analogous change of a percentage of glandular to fat tissues of the imaged subject 110 (See FIG. 1). As an alternative, FIG. 5 illustrates another embodiment of a calibration phantoms 250 can be comprised of contrast agent inserts 251 of different thicknesses (as illustrated) located at different thicknesses of a PMMA material 252 that are known to exhibit image acquisition characteristics analogous to different percentages of glandular to fatty tissue of the imaged subject 110 (See FIG. 1). The phantom 250 also includes a series of other material compositions 253 and 254 different than the PMMA material 252. The material 253 is of a material composition representative or equivalent in radiological attenuation to tissue of zero percent glandular tissue (e.g., one-hundred percent fatty tissue). The material 254 is of a material composition representative or equivalent in radiological attenuation to a tissue of 100 percent glandular tissue. Of course, the location and number of types of tissues 252, 253 and 254 can vary. Consequently, the above-described embodiments of the phantom 230 can be configured with different zones or regions to represent equivalent different thicknesses of breast tissue, and comprised of different types of material composition percentage of glandular to fatty tissue (e.g., zero percent to one-hundred percent glandular tissue), and of different thicknesses of contrast agent 112.

FIG. 4 also illustrates an embodiment of acquired pixel data 255 of a region of interest (ROI) 260 of the calibration phantom 230 as generated by detection of low and high-energy images 265 detected at the array of detectors 125. The acquired pixel data includes, for example, pixel data 270 acquired inside the ROI 260, pixel data 275 acquired outside of the ROI 260, and pixel data 280 acquired along a border defined by a partial thickness 285 of the insert 232 of the contrast agent 112.

FIG. 6 illustrates an example of the detected grayscale levels or value of pixel data acquired in accordance to the act 205 described above mapped or plotted as reference points 305 on a table or graph 310 in logarithmic scale. The calibration act 205 is performed via adjustment of the source 115 and/or controller 120 such images of the phantom 230 are acquired at generally the same low and high energy as anticipated to be used to acquire images of the imaged subject 110. Also assume that detected grayscale levels of pixel data of the material comprising the phantom 230 (e.g., equal to about 40 mm) is generally equal to the grayscale levels of pixel data indicative of the thickness of tissue of the imaged subject 110 (see FIG. 1). Each reference point 305 generally represents a correlation or link of the thickness of the contrast agent 112 relative to detected grayscale values of pixel data of the acquired input images at predetermined low and high spectras of energy of the contrast agent 112 for a type of tissue (i.e., percentage of glandular tissue) and thickness of tissue of the imaged subject 110. The acquired reference points 305 are predetermined to be generally equally spaced apart and selected so as to map or plot in a generally linear manner in the table 310 in relation to axis 315, representative of changing percentage of glandular to fatty tissue, and axis 320, representative of the changing thickness of the contrast agent, respectively. By acquiring and storing the grayscale levels or values of pixel data acquired in the low and high-energy input images for different thicknesses 235 of the calibration phantom 235 (i.e., analogous of the different tissues of the imaged subject 110) in combination with the predetermined thickness 285 of the contrast agent 112 in the calibration phantom 230, the computer 140 is operable to use the table or plot 310 shown in FIG. 6 to calculate the predicted the thickness of the contrast agent 112 in the imaged subject 110.

The computer 140 combines the stored information for predicted grayscale levels or values of pixel data of acquired in both low and high-energy input images of the predetermined thicknesses of the contrast agent 112 in the calibration phantom 235 in combination with the grayscale levels of pixel data in the acquired low and high-energy input images of the imaged subject 110, so as to calculate and create the output image 105 that includes the predicted thickness of the contrast agent 112 in the imaged subject 110, subtracting image noise associated with the thickness of the fat and glandular tissue in the ROI 135 of the imaged subject 110. Accordingly, the computer 140 removes visualization of the texture of the breast tissue from the output image 105, leaving an enhanced illustration of the predicted thickness of the contrast agent 112.

In accordance with one embodiment, the calibrating act 205 is performed for a series of phantoms 230 of different thicknesses 290 and having inserts 232 of the contrast agent 112 of different thicknesses (See FIG. 5). In accordance with another embodiment, the calibrating act 205 is performed with one phantom 250 with different zones of varying thicknesses (See FIG. 5). Accordingly, the above-described series of phantoms 230 or single phantom 250 provides a series of different types and thicknesses of tissue and different contrast agent thicknesses.

The computer 140 controls the energy of the source 115 via the controller 120 in acquiring pixilated image data of the above-described phantom(s) 230 or 250 under the same or similar low and high-energy conditions to be used in acquiring image data of the ROI 135 of the imaged subject 110.

Another embodiment of act 205 includes generating a mathematical model to simulate the predicted thicknesses of the contrast agent 112 injected into the imaged subject 110. One embodiment of the mathematical model representative of a predicted thickness of the contrast agent 112 is in accordance with the following polynomial function:

y=Σ(aᵢⱼ)φ(xₗ)ⁱφ(xₕ)^{j}

where

(y) is a contrast agent thickness,

(xₗ) is a detected grayscale value of pixel data acquired in the low-energy image,

(xₕ) is a detected grayscale value of pixel data acquired in the high-energy image,

(i) and (j) are integers, and

φ(x) is a function of a log-look up table (LUT), analogous to the table 310 of reference points 305 shown in FIG. 6, that maps or correlates acquired grayscale values or levels of pixel data into a radiological thickness domain.

The computer 140 uses the above-described mathematical model and the detected grayscale values of pixel data acquired under low and high-energy so as to calculate and construct a combined image 105 illustrative of the predicted thickness of the contrast agent 112 in the imaged subject 110.

This embodiment of act 205 of generating the mathematical model that simulates a predicted thickness of the contrast agent 112 as a function of grayscale values of pixel data includes determining the coefficients (a_{i,j}) in accordance with the following second order equation (i.e. with six parameters):

y=a_{0,0}+a_{1,0}φ(xₗ)+a_{0,1}φ(xₕ)+a_{1,1}φ(xₗ)φ(xₕ)+a_{0,2}φ(xₗ)²+a_{0,2}φ(xₕ)²

The portion (i.e., a_{0,0}+a_{1,0}φ(xₗ)+a_{0,1}φ(xₕ)) of the above-described mathematical equation generally represents a mathematical model for logarithmic subtraction. It should be understood that other higher order polynomial equations in alternative to the mathematical model described above can be used.

The computer 140 calculates the coefficients (a_{i,j}) through linear regression analysis of the series of reference points (y, xₗ, xₕ) 305, similar to those shown in FIG. 6. The series of reference points (y, xₗ, xₕ) 305 are established by varying a composition of the tissue, and a thickness of the contrast agent 112, and by maintaining the acquisition parameters and the thickness of the tissue at a constant.

In accordance with this embodiment of the simulating act 205, the mathematical model simulates generation of an x-ray energy spectrum given the potential (kVp) and values of parameters representative of the material composition of the radiation generating source 115. For example, assume the data in table 310 as shown in FIG. 6 is generated in accordance with the following values of the energy spectrum: Mo/Mo 25 kV, 100 mAs for the low-energy image acquisition, Mo/Cu 49 kV, 160 mAs for the high-energy image acquisition. The computer 140 is operable to simulate generation of the x-ray spectrum by receiving input or calculating a number of photons generated in the low and high energy spectrums. The model also simulates attenuation of the X-ray energy spectrum through various tissues of various thickness of the imaged subject 110 (e.g., assume a breast thickness of 40 mm), and simulates transformation of the x-ray energy spectrum into a grayscale value of the pixel data detected by the detector 125. Using this above-described simulation mathematical model, the reference points (y, xₗ, xₕ) 305 in FIG. 6 can be simulated, and linear regression analysis of the reference points (y, xₗ, xₕ) 305 is performed to calculate the coefficients (a_{i,j}). Thereby, the coefficients (a_{i,j}) are computed directly adapted to the input low and high-energy spectrum to be used to acquire images of the imaged subject 110.

Once the system 100 has created of generated the mathematical model that calibrates or simulates the predicted thickness of the contrast agent 112, the method 200 includes act 350 of injecting the contrast agent 112 into the imaged subject 110. Act 355 includes acquiring grayscale values of pixel data in the low and high-energy input images of the injected contrast agent 112 in the ROI 135 of the imaged subject 110.

Combining the generated calibration or simulation mathematical model with the acquired pixel data in the low and high-energy images, the method 200 includes an act 360 of generating the output image 105 including an illustration of the predicted thickness of the contrast agent 112 in relation to the ROI 135 of the imaged subject 110 (See FIG. 1).

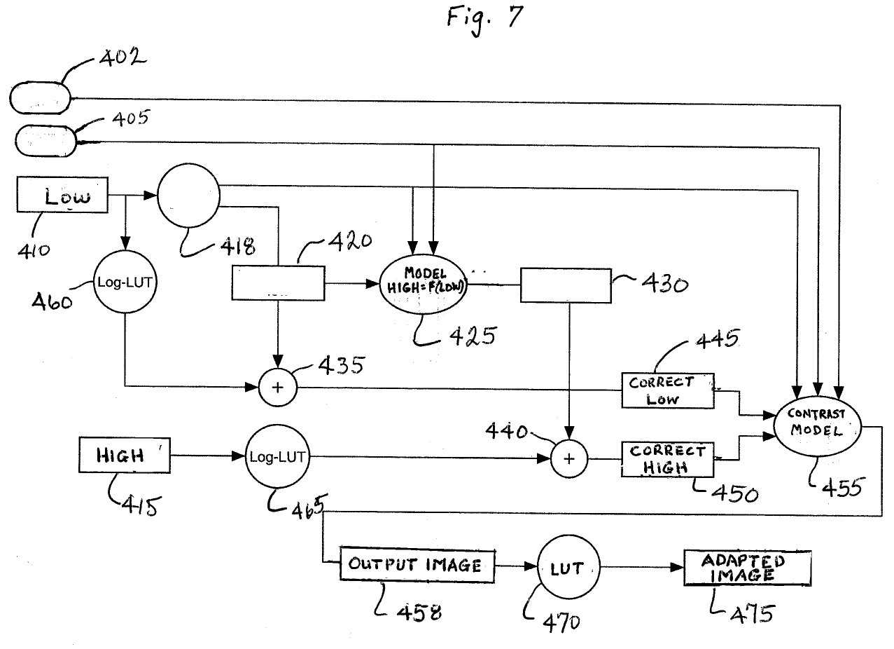

Referring to FIG. 7, assume for sake of example that measured thickness data 402 of the tissue in the ROI 135 of the imaged subject 110, acquisition data 405, and the pixel data in the acquired low and high-energy images 410 and 415 is received at the computer 140. One embodiment of the act 360 generally includes applying a correction at pixel data of the border of the tissue of the imaged subject 110. An embodiment of the correction applying act 360 generally includes act 418 of applying an equalization algorithm in a known manner. An embodiment of the equalization algorithm applying act 418 includes passing the pixel data in the acquired input low-energy image 410 through a low pass filter so as to obtain an image with reduced undesired noise artifacts.

Act 420 includes generating a thick to add correction to the low-energy image 410 in a known manner. The act 420 generally simulates addition or removal of selected image data representative of tissue at a boundary of the ROI 135 so that the full ROI (e.g., breast) 135 can be viewed with a unique width. Using this known technique, a “thick to add” correction is generated and stored which represents the radiological thickness of a layer of one-hundred percent fatty tissue that is added to the input acquired images to achieve a thickness equalization. Also generated and stored is a parameter (θ_{f}), which represents an adipose tissue threshold, such as the grayscale level of fatty tissue in acquired images, computed for the thickness equalization. The value of this parameter (θ_{f}) can be adjusted for a change in an assumption for the thickness of the tissue (e.g., breast tissue) used in the simulation of the reference points so that the simulation of fatty tissue results in the grayscale value (θ_{f}). Accordingly, this parameter (θ_{f}) can be adjusted based on the content of the acquired low and high-energy images.

Act 425 includes generating a model to correlate the acquisition data for the low-energy image 410 with the acquisition data for the high-energy image 415. Act 430 includes applying the model of act 425 in generating a “thick to add” correction for the high-energy image 415. Accordingly, both low and high-energy images 410 and 415 are modified using the generally same added thickness of material via the image chain model of act 425 that gives the grayscale level in the high-energy image 415 as a functional relation of the grayscale level in the low-energy image 410, in the form φ(xₕ)=αφ(xₗ) where (xₕ) and (xₗ) are the grayscale values respectively in the low and high-energy images 410 and 415. The image chain model of act 425 is used to simulate several points (xₗ, xₕ) by varying the tissue thickness while using the acquisition parameters 405 of the input low and high-energy images 410 and 415, respectively. The (α) factor can be computed by linear regression analysis.

Acts 435 and 440 generally include adding the “thick to add” corrections to the low and high-energy images 410 and 415, respectively, creating corrected low and high-energy images 445 and 450, respectively.

Alternatively, a higher-order polynomial expression can be used to generate a functional relation between the grayscale levels in the low-energy image 410 and the grayscale levels in the high-energy image 415.

Once the acquired low and high-energy images 445 and 450 have been corrected through adding of the respective “thick to add” technique, act 455 includes applying the calibration or simulation mathematical model generated in act 205 to the corrected low and high-energy images 445 and 450 so as to generate an output image 458 that includes an illustration representative of a predicted thickness of the contrast agent 112 in relation to the illustration of the tissue in the ROI 135, similar to the output image 105 described above.

Acts 460, 465, and 470 generally includes applying look-up tables (LUTs) to the respective images 410, 415, and 458 so as to create an image adapted with respect to dynamics. As an example, an operator can choose act 470 of applying the LUT to the output image 458 such that a resulting output image 475 fits a 12-bits integer dynamic range. Referring to FIGS. 1 and 7, act 360 includes communicating the output images 458 or 475 for illustration on the display 165.

The system 100 and method 200 described above provides enhanced estimation of a thickness of the contrast agent 112 through the tissue in the ROI 135 under analysis (e.g., mammography of breast tissue) in combination with efficient removal of undesired structure (e.g., breast tissue). Also, the system 100 and method 200 allow for ready calibration adapted to a particular state of the system 100, enhancing accuracy of the predicted thickness of the contrast agent 112.

This written description uses examples to disclose the subject matter described herein, including the best mode, and also to enable any person skilled in the art to make and use the subject matter described herein. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. An imaging system operable to generate an output image of a contrast agent injected into an imaged subject, comprising:
a energy source in communication with a detector, the detector operable to generate a plurality of radiological images of the imaged subject injected with the contrast agent;
a display; and
a computer connected in communication the display and to receive the acquired plurality of images from the detector, the computer including a memory in communication with a processor, the memory including a plurality of programmable instructions for execution by the processor, the plurality of programmable instructions including:
acquiring at least one image of the contrast agent in the imaged subject with a spectra of energy from the energy source;
detecting a plurality of grayscale values of pixel data of the contrast agent in the at least one image;
calculating a predicted thickness of the contrast agent relative to the plurality of grayscale values of pixel data of the contrast agent detected in the at least one image; and
generating an output image comprising an illustration of the predicted thickness of the contrast agent for illustration on the display.

2. The imaging system as recited in claim 1, wherein the act of acquiring the at least one image includes:
acquiring a first image data under a first spectra of energy from the energy source, and
acquiring a second image data under a second spectra of energy from the energy source, the second spectra of energy different than the first spectra of energy.

3. The imaging system as recited in claim 1, the programmed instructions further including:
acquiring a calibration image comprising a plurality of grayscale values of pixel data of the contrast agent having different thicknesses injected in a phantom, the phantom having different thicknesses and different types of material composition.
detecting the plurality of grayscale values of pixel data of the contrast agent in the calibration image at a predetermined spectra of energy; and
generating and storing a table correlating the plurality of grayscale values of pixel data of the contrast agent relative to the predetermined different thicknesses of the contrast agent, the predetermined different thicknesses of the phantom, and at the predetermined spectra of energy for access to in the act of calculating the predicted thickness of the contrast agent,
wherein calculating the predicted thickness includes identifying the predicted thickness of the contrast agent from the table for each of the plurality of grayscale values detected in the at least image of the imaged subject.

4. The imaging system as recited in claim 3, wherein the act of calculating includes interpolating a thickness of contrast agent relative to the table of the plurality of grayscale values of pixel data measured for predetermined different thicknesses of the contrast agent in the calibration image.

5. The imaging system as recited in claim 1, the programmed instructions further including:
acquiring a calibration image comprising a plurality of grayscale values of pixel data of predetermined different thicknesses of the contrast agent injected in predetermined different thicknesses of a phantom and imaged under predetermined different spectras of energy;
calculating and storing a mathematical model representative of a correlation between a grayscale value of pixel data of the contrast agent in the calibration image relative to the predetermined different thicknesses of the contrast agent, the predetermined different thicknesses of the phantom, and the predetermined different spectras of energy; and
inputting the plurality of grayscale values detected in the at least one image of the imaged subject into the mathematical model.

6. The imagine system as recited in claim 5, Wherein the mathematical model that correlates the grayscale values of pixel data of the contrast agent image relative to a thickness of the contrast agent includes:
y=Σa_{i,j}φ(xₗ)ⁱφ(xₕ)^{j}
where
y is the contrast agent thickness,
xₗ the grayscale level in the low-energy image,
xₕ the grayscale level in the high-energy image,
φ(x) is a log-LUT function, and
(a_{i,j}) are coefficients determined through at least one of calibration and simulation.

7. The imaging system as recited in claim 1, further comprising a controller connected to regulate the different levels of spectra of energy emitted by the energy source.

8. The imaging system as recited in claim 7, wherein the computer is connected in communication to regulate the controller.

9. The imaging system as recited in claim 1, wherein the act of acquiring the calibration image includes acquiring images of a plurality of phantoms using predetermined different spectras of energy, each phantom including a plurality of different thicknesses, and a plurality of different thicknesses of the contrast agent inserted in the phantom relative to the other phantoms.

10. A method of generating an output image illustrative of a contrast agent injected into an imaged subject, the method comprising the acts of:
acquiring at least one radiologic image of the imaged subject under a spectra of energy;
detecting a plurality of grayscale values of pixel data of the contrast agent in the first and second images;
calculating a predicted thickness of the contrast agent relative to the plurality of grayscale values of pixel data of the contrast agent detected in the first and second images; and
generating an output image comprising an illustration of the predicted thickness of the contrast agent for illustration on the display.

11. The method according to claim 10, wherein the act of acquiring at least one radiologic image includes:
acquiring a first radiologic image of the imaged subject under a first spectra of energy; and
acquiring a second radiologic image of the imaged subject under a second spectra of energy, the second spectra of energy different than the first spectra of energy.

12. The method according to claim 10, the method further including:
acquiring a calibration image at a predetermined spectra of energy, the calibration image comprising a plurality of grayscale values of pixel data of the contrast agent having different thicknesses injected in a phantom, the phantom having one or more thicknesses and one or more types of material composition representative of a percentage of glandular tissue in a region of interest of the imaged subject;
detecting the plurality of grayscale values of pixel data of the contrast agent in the acquired calibration image; and
generating and storing a table correlating the plurality of grayscale values of pixel data of the contrast agent relative to the predetermined different thicknesses of the contrast agent, the predetermined different thicknesses of the phantom, and at the predetermined spectra of energy for access to in the act of calculating the predicted thickness of the contrast agent,
wherein the act of calculating the predicted thickness includes identifying the predicted thickness of the contrast agent from the table for each of the plurality of grayscale values detected in the at least image of the imaged subject.

13. The method according to claim 12, wherein the act of calculating includes interpolating a thickness of contrast agent relative to the table of the plurality of grayscale values of pixel data measured for predetermined different thicknesses of the contrast agent in the calibration image.

14. The method according to claim 10, the method further comprising the acts of:
acquiring a calibration image comprising a plurality of grayscale values of pixel data of predetermined different thicknesses of the contrast agent injected in predetermined different thicknesses of a phantom and imaged under predetermined different spectras of energy; and
calculating and storing a mathematical model representative of a correlation between a grayscale value of pixel data of the contrast agent in the calibration image relative to the predetermined different thicknesses of the contrast agent, the predetermined different thicknesses of the phantom, and the predetermined different spectras of energy,
wherein the act of calculating the predicted thickness includes inputting the plurality of grayscale values detected in the at least one image of the imaged subject into the mathematical model.

15. The method according to claim 14, wherein the mathematical model that correlates the grayscale values of pixel data of the contrast agent image relative to a thickness of the contrast agent includes:
y=Σ(a_{i,j})φ(xₗ)ⁱφ(xₕ)^{j}
where
y is the contrast agent thickness,
xₗ the grayscale level in the low-energy image,
xₕ the grayscale level in the high-energy image,
φ(x) is a log-Look Up Table (LUT) function, and
(a_{i,j}) are coefficients determined through at least one of calibration and simulation.

16. The method according to claim 15, wherein the coefficients (a_{i,j}) are calculated using a linear regression analysis.

17. The method according to claim 10, further comprising:
regulating differences in the spectra of energy generated by the energy source in acquiring the at least one image of the imaged subject.

18. A calibration phantom to be imaged by a radiological imaging system, comprising:
a main material of at least one thickness; and
at least one insert of a contrast agent of a predetermined thickness located in the main material, the contrast agent operable to be detected in a radiologic image of the calibration phantom.

19. The calibration phantom as recited in claim 18, wherein the phantom includes one a plurality of thicknesses of a material composition, and a plurality of inserts of the contrast agent of a generally uniform thickness located in each of the plurality of thicknesses.

20. The calibration phantom as recited in claim 18, wherein the phantom includes a plurality of material compositions representative of a percentage of glandular tissue of the imaged subject, the plurality of material compositions including a polymethyl methacrylate (PMMA) composition.
